(19) United States
(12) Patent Application Publication
(10) Pub. No.: US 2007/0078520 A1
(43) Pub. Date: Apr. 5, 2007

(54) Traceable nanocrystals and preparation thereof

(75) Inventors: Ying-Jiin Wang, Taipei City (TW); Ching-Shih Lin, Taipei City (TW)

Correspondence Address: HARNESS, DICKEY & PIERCE, P.L.C., P.O. BOX 8910, RESTON, VA, 20195, US

(73) Assignee: National Yang-Ming University

(21) Appl. No.: 11/435,839

(22) Filed: May 18, 2006

(30) Foreign Application Priority Data
Apr. 22, 2005 (CN) 094113007

(51) Int. Cl. A61F 2/28

(52) U.S. Cl. 623/23.51; 623/23.56; 623/908; 600/309

(57) ABSTRACT

Hydroxyapatite (HAp) is widely accepted as bone graft substitutes for clinical application. A novel method is to synthesize an applicable HAp nanoparticles which emit fluorescence or maintain magnetic property with quenching. The present invention is related to utilize CdSe/ZnS or magnetic nano particles as the nuclei for the growth of HAp crystal. The resulted HAp nanoparticles maintain the fluorescent or magnetic characteristics of CdSe QDs or magnetic nanoparticles and exhibit a shell structure of HAp which is recognized by the biological environment. The HAp nanoparticles emit fluorescence or maintain magnetic property which is useful for tracking and could find future clinical applications especially when employed as the tool for in vitro study of bone tissue engineering.

### FIELD OF THE INVENTION

The present invention relates to a method for preparing traceable nanocrystals. The present invention further relates to a nanocomposite comprising traceable nanocrystal for in vivo and in vitro detection or label.

### BACKGROUND OF THE INVENTION

Biomaterials are a class of functional materials designed to interact with and become incorporated into the human body for uses such as prostheses. Unlike products obtained through bioengineering, the manufacture of biomaterials rarely requires cellular processing or a biological intermediary.

There is a need for biomimetic structures friendly to body chemistry and physiology. Goals for these biomaterials are that they possess mechanical stability for hardness, compressive strength, flexural strength, and wear resistance, controlled microstructure to develop functional gradients, controlled interfacial properties to maintain structural integrity in physiological conditions, and well-understood surface chemistry tailored to provide appropriate adhesion properties, chemical resistance, long implant life, and patient comfort.

A wide variety of biomaterials exist such as biocompatible polymers and bioceramics. Biocompatible polymers include biodegradable polymers for use in providing structural support to organs and other body parts, drug delivery, and the like, and non-biodegradable polymers such as polymer prosthesis. For example, hip joint replacements typically make use of non-biodegradable polymers.

Bioceramics have found widespread use in periodontic and orthopedic applications as well as oral, plastic, and ear, nose, and throat surgery. Common materials for bioceramics are alumina, zirconia, calcium phosphate based ceramics, and glass-ceramics. Bioceramics can be categorized according to their in vivo interaction, typically as bioinert, bioactive, and resorbable bioceramics. Various types of bioceramics undergo fixation within the body according to different processes. Some processes are generally more favorable than others, but in many cases a bioceramic material that undergoes fixation within the body via one advantageous interaction may be associated with other disadvantages.

Bioinert bioceramics include single crystal and polycrystalline alumina and zirconia, and are characterized as such because the body encapsulates the ceramics with fibrous tissue as a natural mechanism in recognition of the inert ceramic as a foreign object, and tissue growth associated with this reaction is used to mechanically fix the ceramic article in the body. In dense alumina and zirconia, the tissue grows into surface irregularities. In porous polycrystalline alumina, zirconia, etc., tissue grows into the pores.

Resorbable bioceramics include tricalcium phosphate, calcium sulfate, and calcium phosphate salt based bioceramics. They are used to replace damaged tissue and to eventually be resorbed such that host tissue surrounding an implant made of the resorbable ceramic eventually replaces the implant.

Bioactive bioceramics include hydroxyapatite bioceramics, glass, and glass-ceramics. A “bioactive” material is one that elicits a specific biological response at its surface which results in the formation of a bond with tissue. Thus, bioactive materials undergo chemical reactions in the body, but only at their surfaces. These chemical reactions lead to chemical and biological bonding to tissue at the interface between tissue and a bioactive implant, rather than mere ingrowth of tissue into pores of the implant which provide mechanical fixation. A characteristic of bioactive ceramic articles is the formation of a hydroxycarbonate apatite (HCA) layer on the surface of the article. The degree of bioactivity is measured in terms of the rate of formation of HCA, bonding, strength, and thickness of the bonding layer as well as cellular activity.

Apatite is a calcium phosphate material in crystalline form having the general formula Ca₅ (F, Cl, OH, ½ CO₃) (PO₄)₃. One of the more common types of apatite is hydroxyapatite which has the formula [Ca₂ (PO₄)₂]₃ Ca(OH)₂. It is useful as a packing material to be filled in columns for chromatographic separation of biopolymers such as proteins, enzymes, and nucleic acids. Its ability to adsorb such molecules depends on both the structure of the crystal itself and on the exposed surface area of the crystals.

Problems long associated with resorbable bioceramics are the maintenance of strength, stability of the interface, and matching of the resorption rate to the regeneration rate of the host tissue. Furthermore, the constituents of resorbable biomaterials must be metabolically acceptable since large quantities of material must be digested by cells. This imposes a severe limitation on these compositions.

A composition and structure of hydroxyl apatite (hereinafter referred to simply as “HAp”) are extremely similar to those of bone and tooth as biological inorganic components for constituting an organism body, so that HAp has a high affinity to the organism body. Therefore, HAp has been used as biomaterials, for example, as artificial organ material and medical material for constituting bone and tooth or the like.

There is much room for improvement in the use of HAp as implants. Because (HAp) implants have low reliability under tensile load, such calcium phosphate bioceramics can only be used as powders, or as small, unloaded implants such as in the middle ear, dental implants with reinforcing metal posts, coatings on metal implants, low-loaded porous implants where bone growth acts as a reinforcing phase, and as the bioactive phase in a composite.” Hench reports that HAp has been used as a coating on porous metal surfaces for fixation of orthopedic prostheses, in particular, that HAp powder in the pores of porous, coated-metal implants would significantly affect the rate and vitality of bone ingrowth into the pores. It is reported that many investigators have explored this technique, with plasma spray coating of implants generally being preferred. Hench reports, however, that long term animal studies and clinical trials of load-bearing dental and orthopedic prostheses suggest that the HAp coatings may degrade or come off. Thus, the creation of new forms of HAp having improved mechanical properties would have significant use, but the results of prior art attempts have been disappointing.

Recently, attention has been focused on nanocrystalline or nanocomposite materials for mechanical, optical and catalytic applications. By designing materials from the cluster level, crystallite building blocks of less than 10 nm are possible, through which unique size-dependent properties such as quantum confinement effect and superparamagnetism can be obtained.

Nanometer-scale semiconductor crystallites (known as nanocrystals or quantum dots) (A. P. Alivisatos, Science 271, 933 (1996) could dramatically improve the use of fluorescent markers in biological imaging (M. Bruchez, et al., Science 281, 2013, 1998; W. C. W. Chan, S. Nie, Science 281, 2016, 1998). Because these colloidal particles act as robust, broadly tunable nanoemitters that can be excited by a single light source, they could provide distinct advantages over current in vitro and in vivo markers (e.g., organic dyes and fluorescent proteins). However, before nanocrystals can be widely used as biolabels, they must maintain three properties under aqueous biological conditions: efficient fluorescence, colloidal stability, and low nonspecific adsorption. Unfortunately, despite recent advances (D. Gerion et al., J. Am. Chem. Soc. 124, 7070 (2002), these conditions have not been simultaneously satisfied, limiting the development of in vivo applications of nonaggregated (or individual) semiconductor nanocrystals.

### BRIEF DESCRIPTION OF THE DRAWINGS

FIG. 1 shows hydrophilic quantum dot after changing innate character. (a) illustrates excellent disperse of quantum dot after changing innate character under large scope observation. (b) illustrates the quantum dot is wurtize structure under HRTEM observation on the atom arrangement of hydrophilic quantum dot, wherein a=4.299 nm, c=7.010 nm.

FIG. 2 shows HRTEM illustration after 30 seconds of quantum dot mineralized.

FIG. 3 shows XRD illustration after 30 seconds of quantum dot mineralized.

FIG. 4 shows situation of HAp composite under fluorescent microscope after 10 minutes of quantum dot mineralized. Left figure shows optical microscope without laser excitation. Right figure shows fluorescent illumination from HAp composite under laser inspiration which means the quantum dot inside HAp composite still can be excited.

### DETAILED DESCRIPTION OF THE INVENTION

The present invention relates to biomaterials.

A “biomaterial” is a non-living material used in a medical device which is intended to interact with biological systems. Such materials may be relatively “bioinert”, “biocompatible”, “bioactive” or “resorbable”, depending on their biological response in vivo.

Bioactive materials are a class of materials each of which when in vivo elicits a specific biological response that results in the formation of a bond between living tissue and that material. Bioactive materials are also referred to as surface reactive biomaterials. Biomaterials may be defined as materials suitable for implantation into a living organism. Biomaterials which are relatively inert may cause interfacial problems when implanted and so considerable research activity has been directed towards developing materials which are bioactive in order to improve the biomaterial-tissue interface.

Known bioactive materials include HAp, some glasses and some glass ceramics. Both bioactive glasses and bioactive glass ceramics form a biologically active layer of hydroxycarbonateapatite (HCA) when implanted. This layer is equivalent chemically and structurally to the mineral phase in bone and is responsible for the interfacial bonding between bone and the bioactive material.

Hydroxyapatite, Ca₁₀ (PO₄)₆ (OH)₂, is an attractive and widely utilized bioceramic material for orthopedic and dental implants because it closely resembles native tooth and bone crystal structure. Though HAp is the most common bioceramic, applications for its use have been limited by its processability and architectural design conceptualization. Conventional processing lacks compositional purity and homogeneity. Because HAp is difficult to sinter, dense HAp structures for dental implants and low wear orthopedic applications typically have been obtained by high-temperature and/or high-pressure sintering with glassy sintering aids which frequently induce decomposition to undesirable phases with poor mechanical stability and poor chemical resistance to physiological conditions. Thus, conventionally-formed HAp necessitates expensive processing and compromises structural integrity due to the presence of secondary phases. Existing methods require high forming and machining costs to obtain products with complex shapes. Furthermore, typical conventional HAp decomposes above 1250° C. This results in a material with poor mechanical stability and poor chemical resistance.

While HAp is used widely, and a HAp formulation having mechanical and morphological properties advantageous for prostheses would be very useful, attempts to date have failed to produce reliable structural HAp implants. Accordingly, it is an object of the invention to provide relatively simple techniques for synthesizing nanocrystalline apatite materials having structural and morphological properties useful for structural implants. In particular, it is an object to provide synthesis techniques that produce densified, nanocrystalline material under mild conditions including relatively low sintering temperature, reducing or eliminating decomposition and minimizing cost. It is another object to obtain apatite materials having enhanced mechanical and chemical resistance by maintaining an ultrafine microstructure in sintering through suppression of grain growth.

The definition of nanocomposite material has broadened significantly to encompass a large variety of systems such as one-dimensional, two-dimensional, three-dimensional and amorphous materials, made of distinctly dissimilar components and mixed at the nanometer scale.

The general class of nanocomposite organic/inorganic materials is a fast growing area of research. Significant effort is focused on the ability to obtain control of the nanoscale structures via innovative synthetic approaches. The properties of nanocomposite materials depend not only on the properties of their individual parents but also on their morphology and interfacial characteristics.

Highly luminescent semiconductor quantum dots (zinc sulfide-capped cadmium selenide) have been covalently coupled to biomolecules for use in ultrasensitive biological detection. In comparison with organic dyes such as rhodamine, this class of luminescent labels is 20 times as bright, 100 times as stable against photobleaching, and one-third as wide in spectral linewidth. These nanometer-sized conjugates are water-soluble and biocompatible. Quantum dots that were labeled with the protein transferrin underwent receptor-mediated endocytosis in cultured HeLa cells, and those dots that were labeled with immunomolecules recognized specific antibodies or antigens.

The development of sensitive nonisotopic detection systems has substantially impacted many research areas, such as DNA sequencing, clinical diagnostics, and fundamental molecular biology. These systems aim to solve the problems of radioactive detection (for example, health hazards and short lifetimes) and open new possibilities in ultrasensitive and automated biological assays. Current nonisotopic detection methods are mainly based on organic reporter molecules that undergo enzyme-linked color changes or that are fluorescent, luminescent, or electroactive. A class of nonisotopic detection labels has been developed by coupling luminescent semiconductor quantum dots (QDs) to biological molecules. In this design, nanometersized QDs are detected through photoluminescence, and the attached biomolecules recognize specific analytes, such as proteins, DNA, or viruses. These nanoconjugates are biocompatible and are suitable for use in cell biology and immunoassay. At the present level of development, however, the QDs are not sufficiently monodisperse, and intermittent photon emission could cause statistical problems at the single-dot level.

Fluorescent semiconductor nanocrystals (quantum dots) have the potential to revolutionize biological imaging, but their use has been limited by difficulties in obtaining nanocrystals that are biocompatible. To address this problem, the encapsulated individual nanocrystals in phospholipid block-copolymer micelles and demonstrated both in vitro and in vivo imaging. When conjugated to DNA, the nanocrystal-micelles acted as in vitro fluorescent probes to hybridize to specific complementary sequences. Moreover, when injected into Xenopus embryos, the nanocrystal-micelles were stable, nontoxic (<5×10⁹ nanocrystals per cell), cell autonomous, and slow to photobleach. Nanocrystal fluorescence could be followed to the tadpole stage, allowing lineage-tracing experiments in embryogenesis.

The main challenge is that the quantum dots (QDs), as synthesized, have hydrophobic organic ligands coating their surface (M. A. Hines, P. Guyot-Sionnest, J. Phys. Chem. 100, 468, 1996). To make the QDs water soluble, these organophilic surface species are generally exchanged with more-polar species, and both monolayer (W. C. W. Chan, S. Nie, Science 281, 2016, 1998) and multilayer (M. Bruchez, M. Moronne, P. Gin, S. Weiss, A. P. Alivisatos, Science 281, 2013, 1998) ligand shells have been pursued. Although the monolayer method is reproducible, rapid, and produces QDs with a regular, well-oriented, thin coating, their colloidal stability is poor (J. Aldana, et al., J. Am. Chem. Soc. 123, 8844, 2001). In contrast, the multilayer method yields QDs that are stable in vitro (D. Gerion et al., J. Phys. Chem. B 105, 8861, 2001), but the coating process is long and the coating is difficult to control. A more serious concern is that both approaches still produce QDs that tend to aggregate and adsorb nonspecifically. To resolve this problem, researchers have explored two additional coatings. First, the outer ligand shell of the QD has been overcoated with proteins adsorbed through hydrophobic or ionic interactions (H. Mattoussi et al., J. Am. Chem. Soc. 122, 12142, 2000). Other layers can then be added to allow conjugation with specific biomolecules. Indeed, this method has provided new reagents for fluoroimmunoassays (E. R. Goldman et al., Anal. Chem. 74, 841, 2002). Second, the outer ligand shell has been overcoated with surfactants or polymers to prevent nonspecific adsorption of biomolecules while still permitting bioconjugation. For example, silica-coated QDs have been further modified with small monomers of poly(ethylene glycol) to reduce nonspecific adsorption (D. Gerion et al., J. Am. Chem. Soc. 124, 7070, 2002).

Fluorescence is a widely used tool in biology. The drive to measure more biological indicators simultaneously imposes new demands on the fluorescent probes used in these experiments. For example, an eight-color, three-laser system has been used to measure a total of 10 parameters on cellular antigens with flow cytometry (M. Roederer et al., Cytometry 29, 328, 1997), and in cytogenetics, combinatorial labeling has been used to generate 24 falsely colored probes for spectral karyotyping (E. Schrbck et al., Science 273, 494, 1996).

Metallic and magnetic nanocrystals, with the appropriate organic derivatization of the surface, have been used widely in biological experiments (R. Elghanian, et al., Science 277, 1078, 1997). The use of semiconductor nanocrystals in a biological context is potentially more problematic because the high surface area of the nanocrystal might lead to reduced luminescence efficiency and photochemical degradation. Bandgap engineering concepts borrowed from materials science and electronics have led to the development of core-shell nanocrystal samples with high, room temperature quantum yields (>50%) (X. G Peng, et al., J. Am. Chem. Soc. 119, 7019, 1997) and much improved photochemical stability. By enclosing a core nanocrystal of one material with a shell of another having a larger bandgap, one can efficiently confine the excitation to the core, eliminating nonradiative relaxation pathways and preventing photochemical degradation. The synthesis of the semiconductor nanocrystals and the growth of the shell by methods from the literature yield gram quantities of a variety of materials with a narrow size distribution (5%), coated with a surfactant but soluble only in nonpolar solvents.

Biological applications require water-soluble nanocrystals. The chemistry of the core-shell systems has been extended by adding a third layer of silica that makes the core-shell water soluble, similar to a procedure detailed for coating gold and cadmium sulfide nanocrystals (M. A CorreaDuarte, et al., Chem. Phys. Lett. 286, 497, 1998). This strategy has a number of advantages compared with strategies that use a single direct bond to the surface of the nanocrystal: the multivalency of an extensively polymerized polysilane ensures that the nanocrystals stay soluble in spite of the potential loss of bound thiol.

Quantum dots are usually synthesized in nonpolar organic solvents. If they are to be solubilized in aqueous buffers, their hydrophobic surface ligands must be replaced by amphiphilic ones. Different qdot solubilization strategies have been devised over the past few years, including (i) ligand exchange with simple thiol-containing molecules or more sophisticated ones such as oligomeric phosphines, dendrons, and peptides; (ii) encapsulation by a layer of amphi-philic diblock or triblock copolymers or in silica shells, phospholipids micelles, polymer beads, polymer shells, or amphiphilic polysaccharides; and (iii) combinations of layers of different molecules conferring the required colloidal stability to qdots.

Ceramics of calcium phosphate are the most important implantable materials for repairing bone tissues due to their excellent biocompatibility. Of the various composites of calcium phosphates used today, HAp is widely accepted as bone graft substitutes for clinical application. For both in vitro and in vivo studies of the resorption of HAp, radioactive labeling techniques either with ¹⁶⁶Ho or ¹⁸⁸Re have to be employed. But the main disadvantage of radioactive labeling techniques was the high leakage of radioactivity. An alternative novel approach is to synthesize applicable HAp nanoparticles which emit fluorescence or maintain magnetic property with quenching. The present invention is to utilize CdSe/ZnS or magnetic nano particles as the nuclei for the growth of HAp crystal. The resulted HAp nanoparticles maintain the fluorescent or magnetic characteristics of CdSe QDs or magnetic nanoparticles and exhibit a shell structure of HAp which is recognized by the biological environment. The HAp nanoparticles emit fluorescence or maintain magnetic property which is useful for tracking and could find future clinical applications especially when employed as the tool for in vitro study of bone tissue engineering.

The present invention is related to a method for preparing a nanocrystal emitting fluorescence or maintaining magnetic property comprises:
(a) providing a quantum dot or a magnetic nanoparticle as a nucleus, wherein the nucleus has a functional group with negative charge on the surface of the nucleus, and the functional group with negative charge is bonded with Ca²⁺ from calcium phosphate or sulfate; and
(b) coating the nucleus with various kinds of calcium phosphate or sulfate before quenching.

According to the method of the present invention, the calcium phosphate consists of hydroxyapatite (HAp) and tricalcium phosphate (TCP). But before providing with the nucleus, it should be modified to form carboxyl group on the surface of the nucleus and the carboxyl group is bonded with Ca²⁺ which provided from calcium phosphate or sulfate. This procedure is called chelating reaction. According to the method of the present invention, the HAp or TCP nanocrystal is formed under pH 1˜14. The preferred pH is 4˜8.5. The nanocrystal of the present invention is in size of 40 nm to 10 μm controlled by time in 1 second to 7 days. The fluorescence is emitted by different size of quantum dots. The nanocrystal of the present invention is calcium phosphate or sulfate which is formed under the ratio of 1.0-2.5. The preferred ratio is 1.3-2.0. The most preferred ratio is 1.55-1.75

According to the method of the present invention, the magnetic nanoparticle is selected from the group consisting of cobalt, cobalt alloy, cobalt ferrite, cobalt nitride, cobalt oxide, Co—Pt, Fe, Fe alloy, Fe—Au, Fe—Cu, Fe—N, Iron oxide, Fe—Pd, Fe—Pt, Fe—Zr—Nb—B, Mn—N, Nd—Fe—B, Nd—Fe—B—Nd—Cu, Ni and Ni alloy.

Accordingly, the present invention is also related to a nanocomposite comprising a quantum dot emitting fluorescence or a nanoparticle having magnetism as a nucleus coated with various kinds of calcium phosphate or sulfate. The nanocomposite of the present invention is in size of nanometer or micrometer scale and in shape of particle, bulk, fiber-like, thin film or sponge-like. The quantum dot of the composite is in size of 3 to 20 nm. The nanoparticle having magnetism of the composite which expresses paramagnetism or diamagnetism is in size of 5 to 1000 nm.

The present invention is also related to a composition comprising the nanocomposite described above and a pharmaceutically acceptable carrier.

The present invention is further related to a method for detecting or labeling in vitro or in vivo fluorescence or magnetism comprising administering a patient in need of such detection of the nanocomposite or composition described above. In the method of the present invention, the detection is not limited but to apply cell labeling, artificial bone, bone grafting materials, bone densitometry or scaffold of tissue engineering, especially in the patient suffering from bone disease including rheumatoid arthritis.

The examples below are non-limiting and are merely representative of various aspects and features of the present invention.

### EXAMPLE

Synthesis, Hydrophile and Mineralization of the Quantum Dot or Magnetic Nanocrystal

The synthetic method developed by Bawendi et al. (Bawendi et al., J. Phys. Chem. B, 101, 9463-9475, 1997) for organic metal synthesis to prevent CdSe from oxidizing. The outside of CdSe covered with a layer of ZnS which proceeded as slowly added 1 ml ZnS stock solution onto CdSe at 140° C., then cooled until 100° C. Terminated the reactioin after heating 1 hour. In order to let the quantum dot or magnetic nanocrystal have reactive functional group which used for precipetated calcium phosphate nanocrystal (HAp or TCP) later, 0.01 mmol CdSe/ZnS crystal has been solved in 1 ml chloroform solution, and added 0.5 ml DMF solution and 1 ml overdose Mercaptoacetic acid to react thoroughly by stir vigorously for 1 day at room temperature. At this time, the quantum dot or magnetic nanocrystal has already had carboxyl group.

The quantum dot or magnetic nanocrystal having carboxyl group was soaked in 2 M CaCl₂ (0.5 ml) to react half hour to form linkage between Ca²⁺ and carboxyl group on the surface of the quantum dot or magnetic nanocrystal which called chelating reaction. Supernatant was removed after centrifuge and added 1 ml simulated body fluid (SBF, Na⁺ 142.0 mM, K⁺ 5.0 mM, Mg⁺ 1.5 mM, Ca⁺ 2.5 mM, Cl⁻ 148.8 mM, HCO³⁻ 4.2 mM, HPO₄²⁻ 1.0 mM, SO₄²⁻ 0.5 mM). Different size of calcium phosphate nanocrystals were taken out by time control. The solution contained nanocrystal which been removed was immediately frozen by liquid nitrogen to prevent calcium phosphate nanocrystal from growing. The cooled nanocrystal was preserved in triple distilled water after freeze and dryness to proceed with analyze.

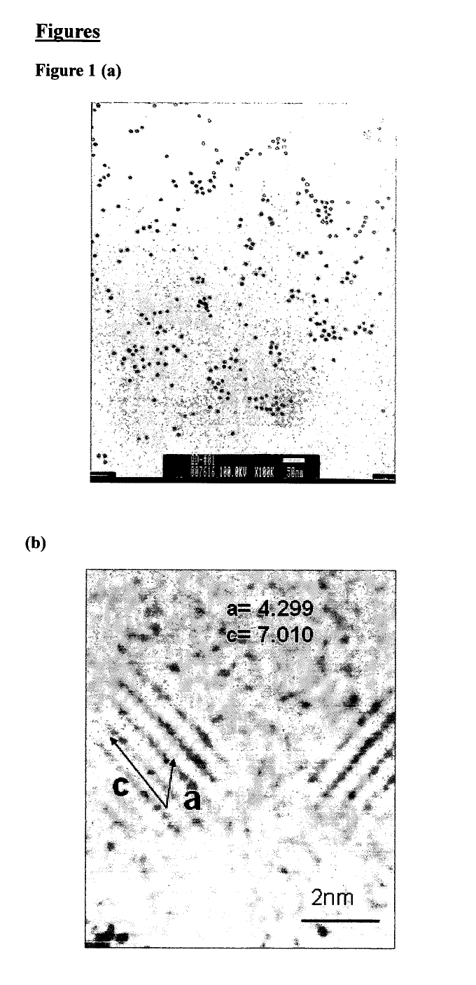

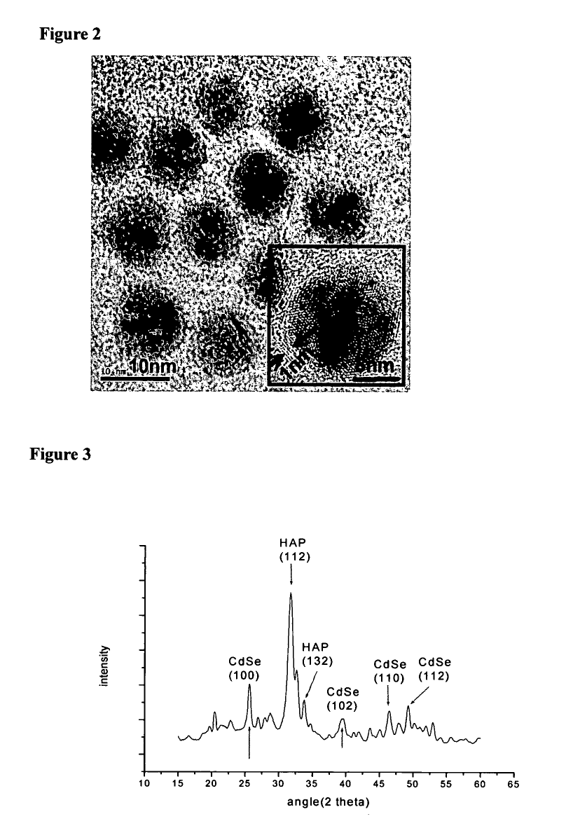

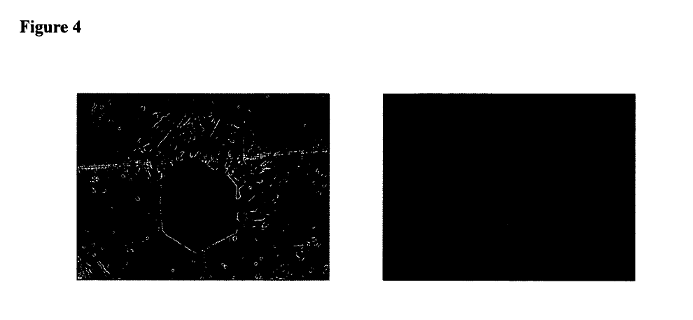

The quantum dot after changing innate character by mercaptoacetic acid was observed its disperse and atom structure by HR-TEM. FIG. 1(a) showed the excellent disperse of the quantum dot, (b) showed it was wurtize structure, wherein a=4.299 nm, c=7.010 nm. If the surface of the quantum dot sinked in HAp crystal for 60 seconds, a thin layer about 1.5 nm could be formed on its surface (FIG. 2). The ratio of Ca/P is about 1.66 by EDX semi-quantify the ingredient of the crystal. In addition, if froze and dried the crystal that mineralized for 60 seconds, the result of powder XRD can express the lattice structure of nano-compound having HAP and quantum dot (FIG. 3). If prolonged the sedimentation of HAp to 10 minutes, the HAp crystal not only self-assembled as hexagonal-like HAp lattice structure but also retained the fluorescent characteristic of the nuclei being quantum dot. FIG. 4 showed the phenomenon of fluorescence illuminated by HAp composite under excitation. It demonstrated that HAp crystal in size of nanometer to micrometer scale had been synthesized.

One skilled in the art readily appreciates that the present invention is well adapted to carry out the objects and obtain the ends and advantages mentioned, as well as those inherent therein. The processes and methods for producing nanoparticles are representative of preferred embodiments, are exemplary, and are not intended as limitations on the scope of the invention.

Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for preparing a nanocrystal emitting fluorescence or maintaining magnetic property comprises:
(b) providing a quantum dot or a magnetic nanoparticle as a nucleus, wherein the nucleus has a functional group with negative charge on the surface of the nucleus, and the functional group with negative charge is bonded with Ca²⁺ from calcium phosphate or sulfate; and
(c) coating the nucleus with various kinds of calcium phosphate or sulfate before quenching.

2. The method according to claim 1, wherein calcium phosphate consisting of hydroxyapatite (HAp) or tricalcium phosphate (TCP).

3. The method according to claim 1, wherein the functional group with negative charge is carboxyl group.

4. The method according to claim 1, wherein the nanocrystal is formed under pH 1˜14.

5. The method according to claim 4, wherein the nanocrystal is formed under pH 4˜8.5.

6. The method according to claim 1, wherein the nanocrystal is in size of 40 nm to 10 μm controlled by time in 1 second to 7 days.

7. The method according to claim 1, wherein the fluorescence is emitted by different size of quantum dots.

8. The method according to claim 1, wherein the calcium phosphate is formed under the ratio of 1.0-2.5.

9. The method according to claim 8, wherein the calcium phosphate is formed under the ratio of 1.3-2.0.

10. The method according to claim 9, wherein the calcium phosphate is formed under the ratio of 1.55-1.75.

11. The method according to claim 1, wherein the magnetic nanoparticle is selected from the group consisting of cobalt, cobalt alloy, cobalt ferrite, cobalt nitride, cobalt oxide, Co—Pt, Fe, Fe alloy, Fe—Au, Fe—Cu, Fe—N, Iron oxide, Fe—Pd, Fe—Pt, Fe—Zr—Nb—B, Mn—N, Nd—Fe—B, Nd—Fe—B—Nd—Cu, Ni and Ni alloy.

12. A nanocomposite comprising a quantum dot emitting fluorescence or a nanoparticle having magnetism as a nucleus coated with various kinds of calcium phosphate or sulfate.

13. The nanocomposite according to claim 12, which is in size of nanometer to micrometer scale.

14. The nanocomposite according to claim 12, which is in shape of particle, bulk, fiber-like, thin film or sponge-like.

15. The nanocomposite according to claim 12, wherein the quantum dot is in size of 3 to 20 nm.

16. The nanocomposite according to claim 12, wherein the nanoparticle is in size of 5 to 1000 nm.

17. The nanocomposite according to claim 12, wherein the nanoparticle can express paramagnetism or diamagnetism.

18. A composition comprising the nanocomposite according to claim 12 and a pharmaceutically acceptable carrier.

19. A method for detecting or labeling in vitro or in vivo fluorescence or magnetism comprising administering a patient in need of such detection or labeling of the nanocomposite of claim 12 or the composition of claim 18.

20. The method according to claim 19, wherein the detection is directed to cell labeling, artificial bone, bone grafting materials, bone densitometry or scaffold of tissue engineering.

21. The method according to claim 19, wherein the patient is sick with rheumatoid arthritis.
